(19) United States
(12) Patent Application Publication
(10) Pub. No.: US 2008/0125744 A1
(43) Pub. Date: May 29, 2008

(54) Drug Delivery Device

(75) Inventor: Kevin Treacy, Galway (IE)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR, INC., SANTA ROSA, CA (US)

(21) Appl. No.: 11/720,535

(22) PCT Filed: Dec. 1, 2005
(86) PCT No.: PCT/IE05/00134

§ 371 Date: May 31, 2007

(30) Foreign Application Priority Data
Dec. 1, 2004 (IE) S2004/0803

(51) Int. Cl. A61M 25/09

(52) U.S. Cl. 604/506; 604/528

(57) ABSTRACT

A drug delivery device comprising an elongate flexible tube having a proximal end and a distal end, the elongate flexible tube being adapted to be slidably movable in a lumen of a catheter and guide wire exchange System; the elongate flexible tube having a lumen extending therethrough, the lumen of the tube being open at the proximal end and distal end and the lumen being adapted to receive liquid under pressure; the drug delivery device also comprising means for engaging the drug delivery device with the catheter and guide wire exchange System, whereby, in use, the drug delivery device is in fluid communication with the catheter and guide wire exchange System and a medication in liquid form is delivered from a reservoir through the elongate flexible tube to the distal end thereof for delivery of the medication to a desired site.

### FIELD OF THE INVENTION

The present invention relates to a drug delivery device. In particular the present invention relates to a drug delivery device for delivering a drug into a patient's vascular system which is to be used in conjunction with a catheter and guide wire exchange system, for example, with the MULTI-EXCHANGE™ catheter and guide wire exchange system described in applicant's patent application PCT/IE03/00052.

### BACKGROUND OF THE INVENTION

Blood vessel narrowing in human body is caused by the deposit of cholesterol and other fatty substances on the inner lining of the blood vessel. Such constriction of a blood vessel is termed a stenosis. Atherosclerosis is a disease arising from a stenosis of a heart artery which leads to serious heart disorders such as ischemic heart disease, stroke and cerebrovascular disease. A number of mechanical procedures is known so far to eliminate artery blockages. Percutaneous catheter procedures are generally less stressful to the patient in comparison to surgical intervention. Such procedure involving the use of catheters for treatment of a vascular constriction is named percutaneous catheter intervention (PCI).

Percutaneous transluminal angioplasty (PTA) is a type of PCI procedure which typically consists of insertion of a catheter with a distally mounted balloon that can be placed, in a deflated condition, within the stenosis, and then inflated to dilate the narrowed lumen of the blood vessel. The designation PTCA, for percutaneous transluminal coronary angioplasty, is used when the treatment is more specifically employed in vessels of the heart. PTCA is used to open coronary arteries that have been occluded by a build-up of cholesterol fats or atherosclerotic plaque. The balloon at the distal end of the catheteris inflated, causing the site of the stenosis to widen.

A PTCA procedure is typically followed by a medication treatment of the lesion in order to prevent possible re-stenosis of the dilated blood vessel, which may occur in a certain amount of cases. Re-stenosis of the treated blood vessel may be caused by thickening of the inner vessel wall due to the growth of smooth muscle cells within the vessel wall. Such cell growth is considered to be a response of the vessel wall tissue to the tissue trauma caused by the dilation device.

One treatment used to prevent or reduce the risk of re-stenosis comprises delivering a drug, which suppresses the growth of smooth muscle cells directly to the treatment site in the blood vessel. Alternatively, a drug can be injected into a peripheral vein for transportation to the treatment site in the blood stream. Direct delivery to the treatment site is preferred as it allows a smaller amount of medicament of higher concentration to be employed in the treatment procedure which contributes to reduction of undesirable side effects on the patient. Other drugs, such as anticoagulants or blood thinning drugs are also commonly delivered during PTCA procedures.

In order to facilitate the PTCA procedure and a subsequent drug delivery procedure, it is advantageous for a PTCA catheter to be designed in such a way as to enable a liquid medication to be transported to the treatment site via a duct in the catheter during or after the PTA/PTCA procedure. Such an arrangement of a PTCA catheter eliminates the need for a complicated procedure of withdrawing the catheter from the patient's vascular system and replacing it with a separate drug delivery device.

A popular type of such PTCA catheter which, along with its function to dilate the stenosed site in the artery, allows a medication to be infused directly to the stenosed site is the so-called “over-the-wire” (OTW) catheter. The placement of such an OTW catheter involves the use of a guide wire, which may be inserted into the patient's vasculature through the skin, and advanced to the location of the treatment site. The catheter, which has a lumen adapted to receive the guide wire, is then advanced over the guide wire. Typically, the guide wire lumen of an OTW catheter extends the entire length of the catheter. The guide wire is disposed entirely within the catheter guide wire lumen except for the distal and proximal portions of the guide wire, which extend beyond the distal and proximal ends of the catheter respectively. As shown in FIGS. 1A and 2A, an OTW catheter 10 typically has a “co-axial” catheter construction, wherein two hollow tubes are nested together such that the lumen 17 of the inner tube can slidably receive a guide wire 15 and the annular luminal space 19 formed between the inner and outer tubes is used for inflation/deflation fluid. An alternative “multilumen” OTW catheter construction has an elongate shaft 10′ made from a single extruded tube having two lumens 17′ and 19′ formed side-by-side, as shown in FIGS. 1B and 2B. OTW catheters that contain both multilumen segments and coaxial segments are also known. Drug infusion in OTW catheters is generally carried out via the full-length guide wire lumen which can also be used for transporting radiocontrast dye to the stenosed artery, for making pressure measurements, and for other therapies. The expandable dilation balloon at the distal end of the catheter may have a plurality of pores through which the medication is ejected onto the treatment area of the blood vessel, when the balloon is inflated. In this instance, the medication liquid is delivered via the inflation fluid lumen and acts as the dilating fluid.

Over-the wire catheters have many advantages attributable to the presence of a full length guide wire lumen. Such advantages include good stiffness characteristics and pushability for readily advancing the catheter through the tortuous vasculature and across tight stenoses. Finally, the full length guide wire lumen permits removal and replacement of a guide wire in an indwelling catheter, as may be required to alter the shape of the guide wire tip. It is also sometimes desirable to exchange one guide wire for another guide wire having a different stiffness. For example, a relatively soft, or flexible guide wire may prove to be suitable for guiding a PTCA catheter through a particularly tortuous anatomy, whereas following up with a stent-delivery catheter through the same vasculature region may require a guide wire that is relatively stiffer.

Over the wire catheters do suffer some shortcomings, however. For example, it often becomes necessary, in the performance of a PCI, to exchange one indwelling catheter for another catheter. In order to maintain a guide wire in position while withdrawing the catheter, the guide wire must be gripped at its proximal end to prevent it from being pulled out of the blood vessel with the catheter. For example, a PTCA catheter, which may typically be of the order of 135 centimeters long, is longer than the proximal portion of the standard guide wire that protrudes out of a patient. Therefore, exchanging an over the wire PTCA catheter requires an exchange guide wire of about 300 centimeters long, whereas a standard guide wire is about 165 centimeters long.

In one type of over the wire catheter exchange, the standard length guide wire is firstly removed from the lumen of the indwelling catheter. Then, a longer exchange guide wire is passed through the catheter to replace the original wire. Next, while holding the exchange guide wire by its proximal end to control its position in the patient, the catheter is withdrawn proximally from the blood vessel over the exchange guide wire. After the first catheter has been removed, the next OTW catheter is threaded onto the proximal end of the exchange guide wire and is advanced along the exchange guide wire, through the guiding catheter, and into the patient's blood vessels until the distal end of the catheter is at the desired location. The exchange guide wire may be left in place or it may be exchanged for a shorter, conventional-length guide wire. In an alternative type of catheter exchange procedure, the length of the initial guide wire may be extended by way of a guide wire extension apparatus. Regardless of which exchange process is used, the very long exchange guide wire is awkward to handle, thus requiring at least two operators to perform the procedure.

A catheter designed to eliminate the need for the use of long exchange guide wires or guide wire extensions has been designed by the applicant and disclosed in applicant's patent application PCT/IE03/00052. The applicant's MULTI-EXCHANGE™ catheter and guide wire exchange system as described in the specification of PCT/IE03/00052 overcomes the foregoing difficulties and is particularly suitable for PTCA procedures involving an exchange of either guide wire or catheter.

The applicant's MULTI-EXCHANGE™ catheter and guide wire exchange system, as shown in FIGS. 3 to 8 of the accompanying drawings comprises an elongate flexible catheter 30 on which a guide member 32 is slidably mounted. The catheter 30 has proximal and distal ends 37 and 38, respectively, and first and second lumens 46 and 42 respectively, extending through the catheter 30. The first lumen 46 is open at the catheter 30 distal end 38 and is sized and shaped to slidably receive a guide wire 15. The portion of the guide wire 15 which is located distally of the guide member 32 (to the right as seen in FIG. 3) is contained and housed within the catheter 30 except for the distal end 36 of the guide wire 15 which may protrude out of the distal end 38 of the catheter 30. The guide member 32 is slidably mounted on the catheter shaft 30 and is received in a guide way 48 formed from a longitudinal cut in the catheter shaft 30 to enable transverse access to the first lumen 46 through the catheter shaft 30. The guide way 48 extends along a major portion of the length of the catheter shaft 30 from a location adjacent the proximal end 37 of the catheter to a location proximal of the shaft distal end 38. Thelongitudinal guide way 48, when the catheter 30 is viewedin cross-section, as in FIGS. 4 to 6, may be considered as defining a pair of flaps 50 which normally close together at the guide way 48 to define enclosed guide wire lumen 46. An elongate stiffening member 43 is disposed within the second lumen 42 from the shaft proximal end 37 to a location adjacent the guide way distal end 52. A balloon 40 is mounted about the shaft distal segment 34, the balloon 40 being in fluid communication with the second lumen 42 so that the second lumen 42 functions as an inflation lumen 42. The inflation lumen 42 extends from the proximal end 37 of the catheter 30, where it communicates with a fitting 44 and extends the length of the catheter 30, terminating in communication with the interior of the balloon 40. As shown in FIG. 8, the guide member 32 has a catheter passageway 62 extending therethrough for slidably receiving the catheter shaft 30 and a guide wire passageway 66 for slidably receiving the guide wire 15. The guide wire passageway 66 intersects the catheter passageway 62 for merging the guide wire 15 and the catheter 30 by guiding the guide wire 15 transversely through the guide way 48 in the catheter and into the first lumen 46. Conversely, the guide member 32 can be used for separating the guide wire 15 and the catheter 30 by guiding the guide wire 15 transversely out of the first lumen 46 through the guide way 48.

In the MULTI-EXCHANGE™ catheter shown in FIGS. 3 and 8, the distal end 52 of guide way 48 terminates short of the distal end 38 of the catheter 30, thereby leaving the distal segment 34 of the catheter 30 in which the guide wire lumen 46 is defined by a continuous surrounding wall 49 as shown in FIG. 7. Adjacent the guide way distal end 52, the shaft of the catheter 30 may transform from the more proximal side-by-side arrangement of lumens to the more distal coaxial arrangement, as will be understood by those skilled in the art. The distal segment 34 preferably comprises a coaxial arrangement of two tubes, as shown in FIG. 7 with the inner tube wall 49 communicating with and surrounding an extension of the guide wire lumen 46. The outer tube 51 encompasses the inner tube, forming an annular lumen that extends the inflation lumen 42 from the region of the guide way distal end 52 to the balloon 40.

When using the MULTI-EXCHANGE™ catheter 30 (as shown on FIG. 8a), the guide wire 15 is manoeuvered through the patient's vascular system such that the distal end 36 of the guide wire 15 is positioned across the treatment site. With the guide member 32 positioned near the distal end 38 of the catheter 30, the proximal end 35 of the guide wire 15 is threaded into the opening of guide wire lumen 46 at the distal end 38 of the catheter 30 and through the guide member 32, such that the proximal end 35 of the guide wire 15 protrudes out the proximal end of the guide member 32. By securing the guide member 32 and the proximal end 35 of the guide wire 15 in a fixed position, the catheter 30 may then be transported over the guide wire 15 by advancing the catheter 30 toward the guide member 32. In doing so, the catheter 30 advances through the guide member 32 such that the guide wire lumen 46 envelops the guide wire 15 as the catheter 30 is advanced into the patient's vasculature. In a PTCA embodiment, the MULTI-EXCHANGE™ catheter 30 maybe advanced over the guide wire 15 in this manner until the distal end 38 of the catheter 30 having the dilation balloon 40 is positioned within the stenosis and essentially the entire length of the guide wire 15 is encompassed within the guide wire lumen 46.

Furthermore, the indwelling MULTI-EXCHANGE™ catheter may be exchanged with another catheter by reversing the operation described above. To this end, the indwelling catheter may be removed by withdrawing the proximal end of the catheter from the patient while holding the proximal end of the guide wire and the guide member in a fixed position. When the catheter has been withdrawn to the point where the distal end of the cut has reached the guide member, the distal portion of the catheter over the guide wire is of a sufficiently short length that the catheter maybe drawn over the proximal end of the guide wire without releasing control of the guide wire or disturbing its position within the patient. After the catheter has been removed, another MULTI-EXCHANGE™ catheter maybe threaded onto the guide wire and advanced over the guide wire in the same manner described above with regard to the MULTI-EXCHANGE™ catheter. The MULTI-EXCHANGE™ catheter permits catheter exchange without the use of the very long exchange guide wire and without requiring withdrawal of the initially placed guide wire.

The zipper type catheter, however, does not feature the possibility of using the guide wire lumen of the catheter for drug infusion or for delivery of radiocontrast dye or other liquids due to a non-continuous structure of the wall guide wire lumen, which has a longitudinally cut guide way substantially along the entire length of the guide wire lumen.

Considering the aforementioned advantages of the MULTI-EXCHANGE™ catheter it is therefore an object of the present invention to equip such a catheter with a means for delivering a drug directly to the site to be treated in a blood vessel during or after a PTA or a PTCA procedure.

### SUMMARY OF THE INVENTION

The present invention accordingly provides a drug delivery device comprising an elongate flexible tube having a proximal end and a distal end, the elongate flexible tube being adapted to be slidably movable in a lumen of a catheter and guide wire exchange system; the elongate flexible tube having a lumen extending therethrough, the lumen of the tube being open at the proximal end and distal end and the lumen being adapted to receive liquid under pressure; the drug delivery device also comprising means for engaging the drug delivery device with the catheter and guide wire exchange system, whereby, in use, the drug delivery device is in fluid communication with the catheter and guide wire exchange system and a medication in liquid form is delivered from a reservoir through the elongate flexible tube to the distal end thereof for delivery of the medication to a treatment site.

Preferably, the elongate flexible tube is adapted to be slidably moveable in a guide wire lumen of the catheter and guide exchange system.

Ideally, the catheter and guide wire exchange system includes a longitudinal guide way formed in the catheter shaft to enable transverse access to the lumen of the catheter, the guide way extending along a major portion of the length of the catheter shaft from a location adjacent the shaft proximal end to a distal terminal end proximal of the catheter shaft distal end, thereby defining an uncut distal segment of the catheter shaft.

The drug delivery device may include a reservoir for storing medication to be delivered to a treatment site of a patient.

Conveniently, in use, the elongate flexible tube of the drug delivery device is introduced through a guide member of the catheter and guide wire exchange system.

The means for engaging the drug delivery device with the catheter and guide wire exchange system preferably comprises a handle mounted on the proximal end of the tube and adapted for engagement with a handle of the catheter and guide wire exchange system.

The handle of the drug delivery device ideally includes a recess which is correspondingly sized and shaped to engageably receive a portion of the handle of the catheter.

Advantageously, the handle of the drug delivery device and the handle of the catheter can be resiliently formed so that the handles are connected together by snap-fitting the catheter handle and the drug delivery device handle together.

The present invention further provides a method ofpreparing a liquid medicament for delivery, comprising the following steps:
(a) loading, via a guide member, a distal end of a drug delivery device comprising an elongate flexible tube into a lumen of a catheter and guide wire exchange system and advancing the distal end of the elongate flexible tube towards the distal end of the catheter;
(b) advancing the tube until a handle of the drug delivery device and handle of the catheter are aligned side by side each other,
(c) connecting the drug delivery device to the catheter by connecting the handle of the drug delivery device with the handle of the catheter, and
(d) providing a reservoir for liquid medicament and directing medicament from the reservoir to the distal ends of the drug delivery device and catheter.

Ideally at step (a) the elongate flexible tube is loaded into a guide wire lumen of the catheter and preferably between step (c) and step (d), there is included a step of drawing the distal tip of the catheter proximally in order to position the tip of the catheter at a desired site.

The present invention also provides a method of delivering a drug comprising the following steps:

(A) loading, via a guide member, a distal end of a drug delivery device comprising an elongate flexible tube into a lumen of a catheter and guide wire exchange system and advancing the distal end of the elongate flexible tube towards the distal end of the catheter,
(B) advancing the tube until a handle of the drug delivery device and handle of the catheter are aligned side by side each other,
(C) connecting the drug delivery device to the catheter by connecting the handle of the drug delivery device with the handle of the catheter, and
(D) injecting a medication in liquid form from a reservoir through the drug delivery device so that the medicament is delivered through the drug delivery device to the required treatment site.

Ideally at step (A) the elongate flexible tube is loaded into a guide wire lumen of the catheter 30 and preferably between step (C) and step (D), there is included a step of drawing the distal tip of the catheter proximally in order to position the tip of the catheter at a desired site.

The method preferably also includes the following step: after injecting the medication using the drug delivery device at step (D) above, flushing the device with saline solution and injecting the saline solution through the drug delivery device to ensure all the medication has reached the treatment site.

### BRIEF DESCRIPTION OF THE DRAWINGS

The invention will now be described more particularly with reference to the accompanying drawings, which show, by way of example only, one embodiment of the drug delivery device of the invention.

In the drawings:

FIG. 1A is a longitudinal sectional illustration of a section of a prior art coaxial over-the-wire catheter and guide wire system;

FIG. 1B is a longitudinal sectional illustration of a section of a prior art multilumen over-the-wire catheter and guide wire system;

FIG. 2A is a transverse sectional illustration of a coaxial prior art over-the-wire catheter and guide wire system, taken along the line 2A-2A of FIG. 1A;

FIG. 2B is a transverse sectional illustration of a multilumen prior art over-the-wire catheter and guide wire system, taken along the line 2B-2B of FIG. 1B;

FIG. 3 is an illustration of an assembly of applicant's MULTI-EXCHANGE™ catheter, guide wire and guide member as discussed above;

FIG. 4 is a transverse sectional illustration of the catheter and guide wire as seen along the line 8-8 of FIG. 3;

FIG. 5 is a transverse sectional illustration of the catheter, guide wire and guide member as seen along the line 9-9 of FIG. 3;

FIG. 6 is a transverse sectional illustration of the catheter, guide member and guide wire as seen along the line 10-10 of FIG. 3;

FIG. 7 is a transverse sectional illustration of the catheter and guide wire as seen along the line 11-11 of FIG. 3;

FIG. 8 is an enlarged longitudinal sectional view of the guide member as seen along the line 12-12 in FIG. 5;

FIG. 8a shows the guide member of FIG. 8, with the guide member positioned at the distal end of the catheter;

FIG. 9 is a side view of the drug delivery device of the invention assembled with the applicant's MULTI-EXCHANGE™ catheter;

FIG. 9a is an enlarged longitudinal sectional view corresponding with that shown in

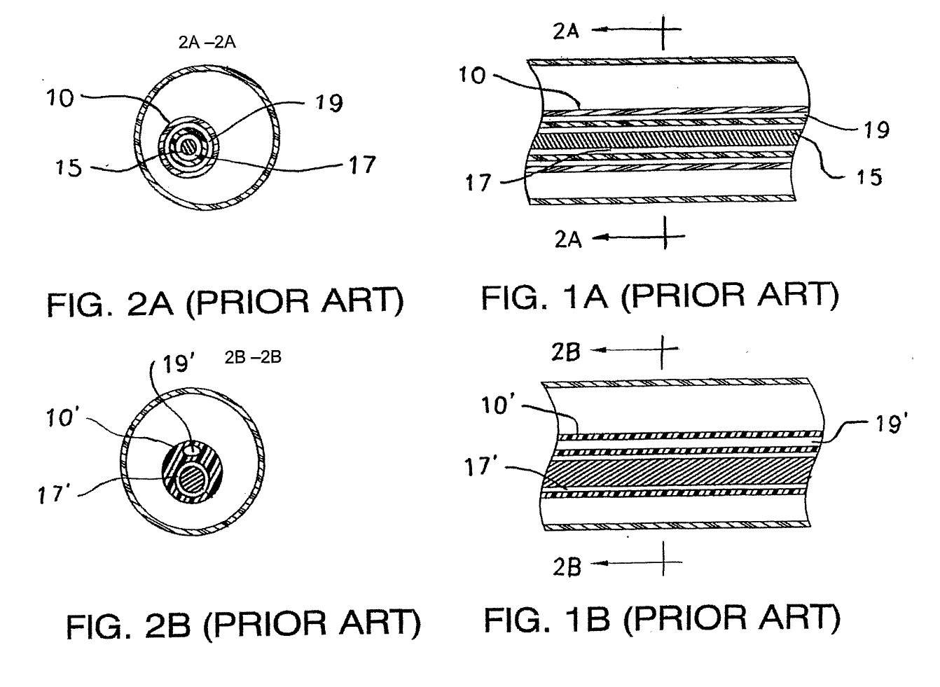

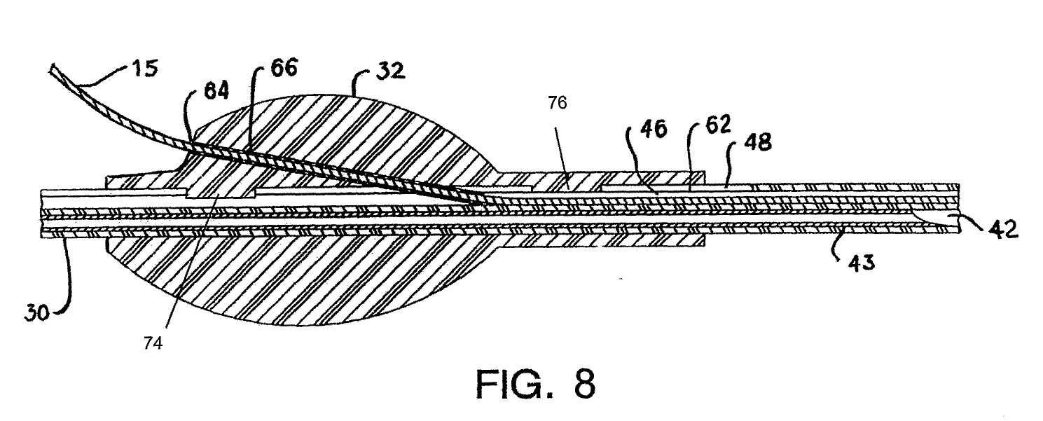

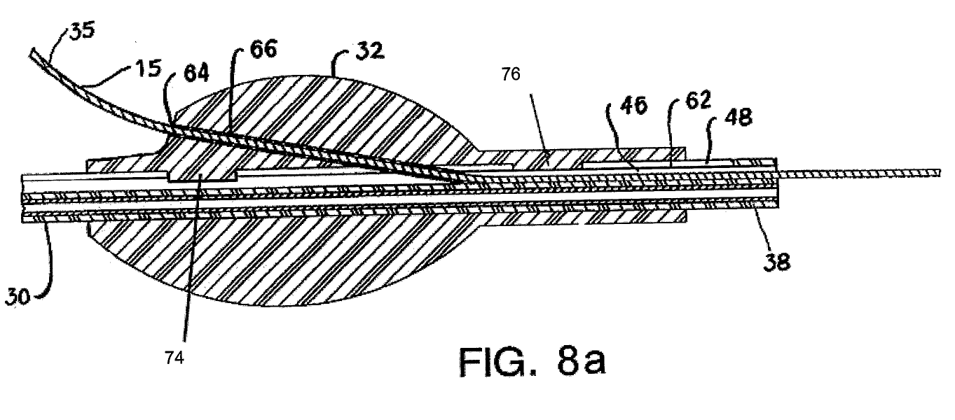

FIG. 8 but showing the tube of the drug delivery device placed through the guide wire lumen of the catheter after the guide wire has been removed therefrom;

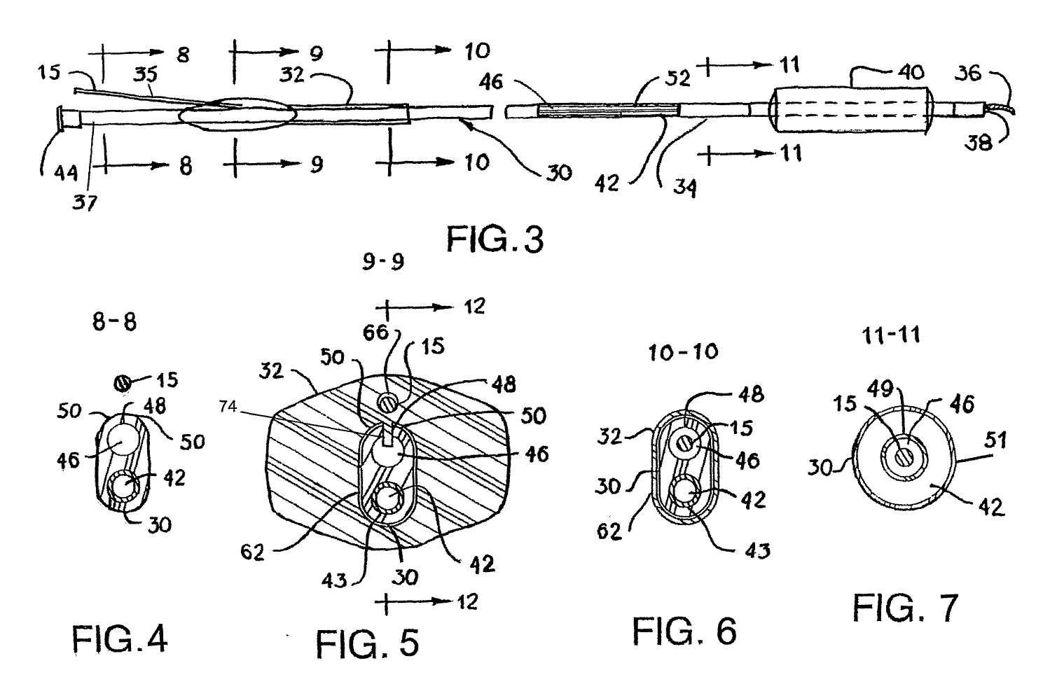

FIG. 10 is a perspective view of the handle of the drug delivery device of the invention clipped together with the handle of the MULTI-EXCHANGE™ catheter shown in FIGS. 3 to 8a thereof;

FIG. 11 is a side elevation of the handle of the drug delivery device of the invention clipped together with the handle of the MULTI-EXCHANGE™ catheter as shown in FIG. 10;

FIG. 11 a is an end cross-sectional elevation as seen along the lines A-A of FIG. 11 of the handle of the drug delivery device of the invention clipped together with the handle of the MULTI-EXCHANGE™ catheter; FIG. 12 is an end elevation of the handle of the drug delivery device of the invention clipped together with the handle of the MULTI-EXCHANGE™ catheter as seen in the direction of arrow X of FIG. 11; FIG. 13 is a plan view of the handle of the drug delivery device of the invention clipped together with the handle of the MULTI-EXCHANGE™ catheter; FIG. 13a is a cross-sectional elevation of the handle of the drug delivery device of the invention clipped together with the handle of the MULTI-EXCHANGE™ catheter as seen along the lines B-B of FIG. 13; FIG. 14 is a perspective view of the handle of the drug delivery device of the invention; FIG. 15 is a side elevation of the handle of the drug delivery device of the invention; FIG. 15a is an end cross-sectional elevation of the handle of the drug delivery device of the invention as seen along the lines C-C of FIG. 15; FIG. 16 is a plan view of the handle of the drug delivery device of the invention; and FIG. 16a is a cross-sectional elevation of the handle of the drug delivery device of the invention as seen along the lines D-D of FIG. 16.

### DETAILED DESCRIPTION OF THE INVENTION

As shown in FIG. 3, the invention includes a catheter, indicated generally by the reference character 30, on which a guide member 32 is slidably mounted. Guide wire 15 is illustrated as extending through the guide member 32. Guide member 32 serves as a juncture in which the catheter 30 and guide wire 15 may be merged or separated so that the portion of guide wire 15 which extends proximally of guide member 32 (to the left as seen in FIG. 3) is separated from catheter 30 and the portion of guide wire 15 which is located distally of guide member 32 (to the right as seen in FIG. 3) is contained and housed within catheter 30 except for distal end 36 of guide wire 15 which may protrude distally out of distal end 38 of catheter 30.

Catheter 30 includes an elongate, flexible, cylindrical main body, which maybe formed from an extruded plastic material such as, for example, polyethylene or polyethylene block amide (PEBA) copolymer. In the embodiment shown in FIG. 3, catheter 30 is a delivery catheter, such as for PTCA or stent delivery, having balloon 40 mounted around the catheter body near the distal end 38 of catheter 30. Balloon 40 may be inflated and deflated through inflation lumen 42 formed through the body of the catheter 30. Inflation lumen 42 extends from the proximal end of catheter 30, where it communicates with fitting 44 and extends the length of catheter 30, terminating in communication with the interior of balloon 40. Fitting 44 may be connected to a suitable source of pressurized fluid or a partial vacuum (not shown) to inflate or deflate balloon 40. Catheter 30 includes another lumen, indicated at 46, which is intended to receive guide wire 15. Guide wire lumen 46 may extend the full length of catheter 30, terminating at distal opening 38 and proximal fitting 44.

The body of catheter 30 is formed with longitudinal guide way 48 which, when catheter 30 is viewed in cross-section, as in FIG. 4, may be considered as defining a pair of flaps 50 which normally close together at guide way 48 to define enclosed guide wire lumen 46. Guide wire lumen 46 may be circular in cross-section or may be non-circular; in either case, the cross-sectional dimensions of guide wire lumen 46 are greater than the cross-sectional dimension of guide wire 15 to permit relative longitudinal movement between guide wire 15 and catheter 30. inflation lumen 42 encompasses elongate stiffening member 43, which causes the shaft of catheter 30 to have greater bending stiffness than guide wire 15.

The proximal end of guide way 48 may terminate at or near fitting 44. In the embodiment shown in FIGS. 3 and 8, distal end 52 of guide way 48 terminates short of distal end 38 of catheter 30, thereby leaving distal segment 34 of catheter 30 in which guide wire lumen 46 is defined by a continuous surrounding wall 49 as shown in FIG. 7. Adjacent guide way distal end 52, the shaft of catheter 30 may transform from the more proximal side-by-side arrangement of lumens to the more distal coaxial arrangement, as will be understood by those of skill in the art. Distal segment 34 preferably comprises a coaxial arrangement of two tubes, as shown in FIG. 11A with inner tube wall 49 communicating with and surrounding an extension of guide wire lumen 46. The outer tube 51 encompasses the inner tube, forming an annular lumen that extends inflation lumen 42 from the region of guide way distal end 52 to balloon 40. Optionally, the distal segment 34 may comprise a multilumen arrangement of the inflation lumen 42 and guide wire lumen 46 as shown in FIG. 6.

Guide member 32 has proximal and distal ends, 54, 56, respectively, as shown in FIGS. 3 and 8. Catheter passageway 62 extends longitudinally in a generally straight line from guide member proximal end 54 to guide member distal end 56. Guide wire passageway 66 extends distally from its end 64, formed at guide member proximal end 54, to intersect catheter passageway 62 at a shallow angle, preferably in a coaxial relationship with guide wire lumen 46. Proximal spreader member 74 is formed in the body of guide member 32 and projects into catheter passageway 62, proximal to the intersection of passageways 62 and 66. Guide member also includes distal spreader member 76, located within guide member distal end 56. Distal spreader member 76 may serve to align catheter 30 within catheter passageway 62, and especially to line up guide way 48 with guide wire passageway 66. Distal spreader member 76 may be disposed adjacent, alongside or spaced from the distal end of guide wire tube 68. As distinguished from proximal spreader member 74, distal spreader member 76 should not project into guide wire lumen 46, where it could interfere with guide wire 15, and longitudinal movement thereof.

Guide member 32 may be molded from a suitable rigid plastic material, such as nylon or nylon based co-polymers that are preferably lubricious. Alternatively, guide member 32 may be made of a suitable metal such as stainless steel or guide member 32 may have both metal components and plastic components. For ease in manufacturing, guide member 32 may be comprised of molded parts that snap-fit together to form the final configuration.

When catheter 30 and guide wire 15 both extend through guide member 32, they merge at the juncture of the passageways as shown in FIG. 8. Entering guide member proximal end 54, catheter 30 extends through catheter passageway 62, engaging spreader 74, which extends through guide way 48 in catheter 30 to spread flaps 50 apart as indicated in FIG. 5. Guide wire 15 may extend from end 64 through guide wire passageway 66 into catheter passageway 62, entering guide wire lumen 46 through spread-apart flaps 50. During advancement of catheter 30 through guide member 32, flaps 50 draw together under the influence of the inherent resiliency of the catheter body to close guide way 48, thus enclosing guide wire 15 within guide wire lumen 46. Guide wire 15 is contained within guide wire lumen 46 from the intersection of passageways 62, 66 within guide member 32 to distal opening 38. The shaft rigidity provided by stiffening member 43 allows catheter 30 to be pushed into guide member proximal end 54 without buckling, despite the lack of guide wire support in this region.

In an alternative manoeuver, guide wire 15 may be inserted or removed through guide wire passageway 66, while guide member 32 is held stationary with respect to catheter 30. In this fashion, guide wire 15 can be exchanged within catheter 30. In yet another type of manipulation, guide wire 15 and catheter 30 can be held relatively still while guide member 32 is translocated, thus unzipping and zipping guide wire 15 and catheter 30 transversely apart or together, depending on which direction guide member 32 is moved. In use, guide member 32 may be secured to a Touhy-Borst or Y-adapter and thus an outer section of guide member 32 may be configured to be received in such an adaptor.

To minimize the amount of material surrounding guide wire lumen 17 and inflation lumen 19, at least the shaft portion of catheter 30 comprising guide way 48 is generally oval in cross-sectional shape, as illustrated in FIGS. 4, 5 and 6. One advantage of such a catheter shape is that the small perimeter, and the correspondingly small area of the cross-section will maximize the surrounding annular space when catheter 30 lies within guiding catheter 5. An additional advantage of the oval cross-sectional shape is that catheter 30 will tend to align itself with catheter passageway 62, which has a matching oval cross-section, as shown in FIGS. 5 and 6. However, proximal shaft section 35 and catheter passageway 62 may also be generally circular.

FIG. 7 illustrates distal section 34 of catheter 30 as having a round cross-sectional shape since it has a coaxial arrangement of the guide wire and inflation lumens. The distal section of catheter could, optionally, have an oval cross section such as shown in 6, regardless of whether or not there is a coaxial or multilumen arrangement of the guide wire and inflation lumens

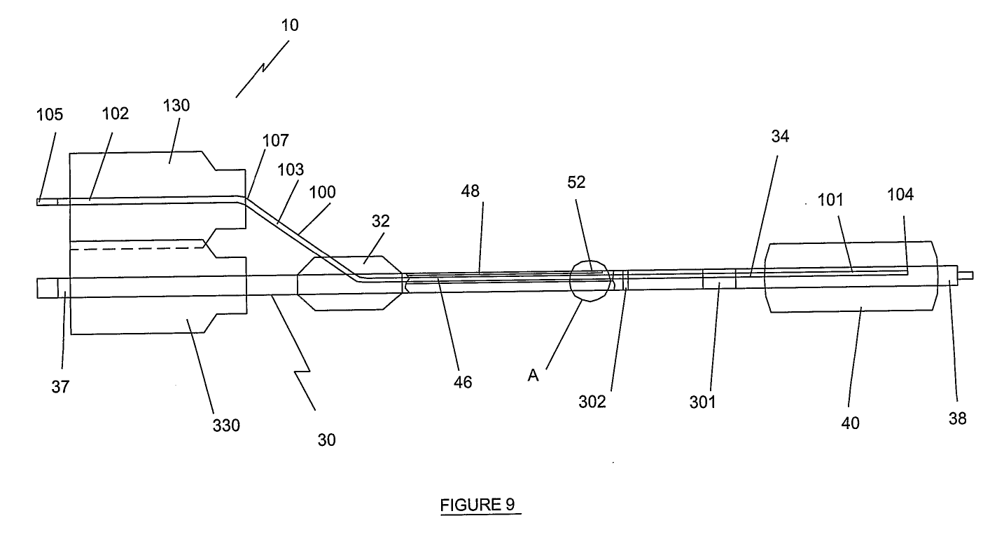

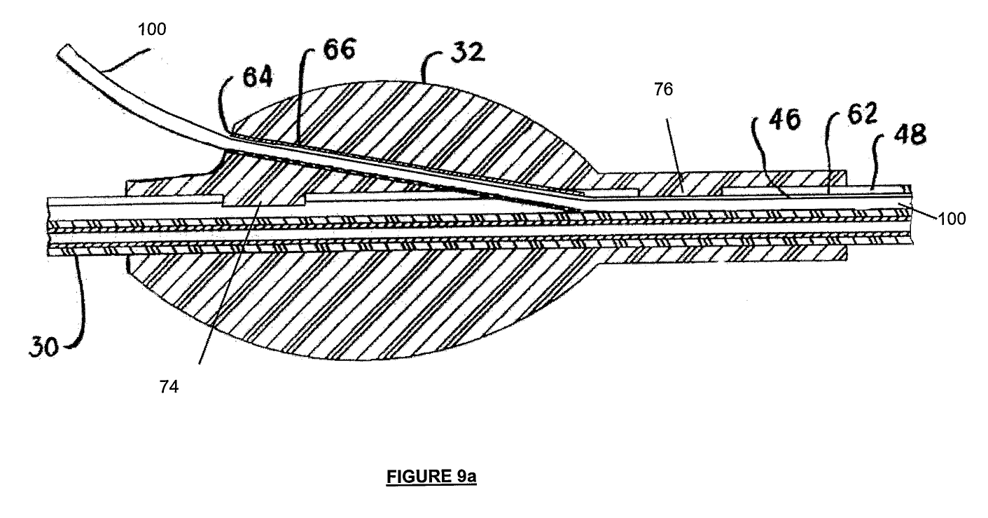

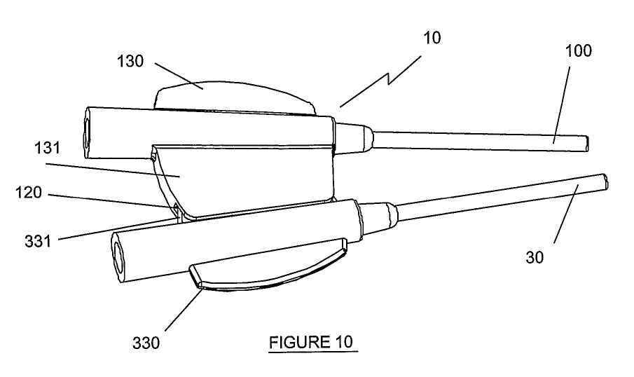

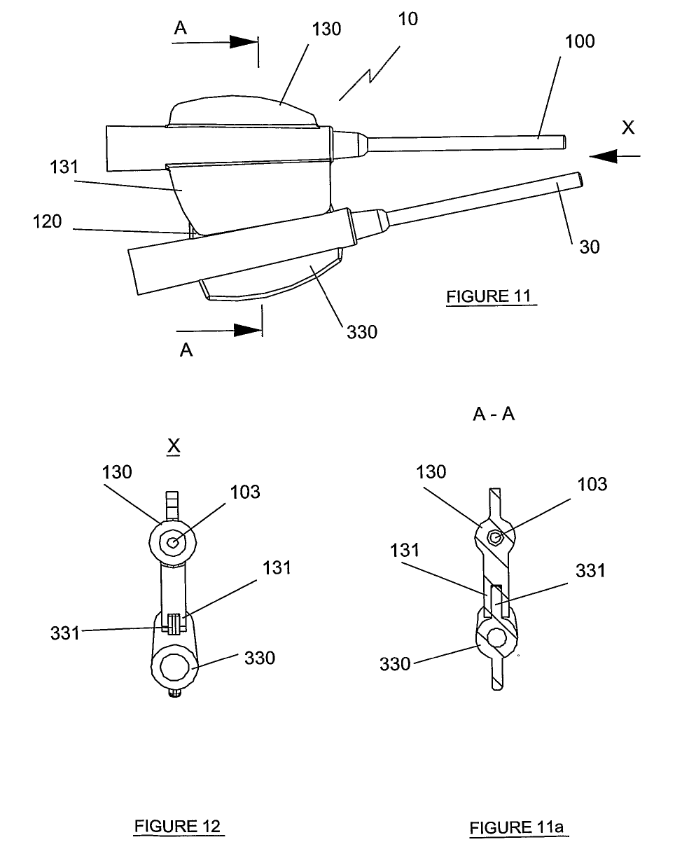

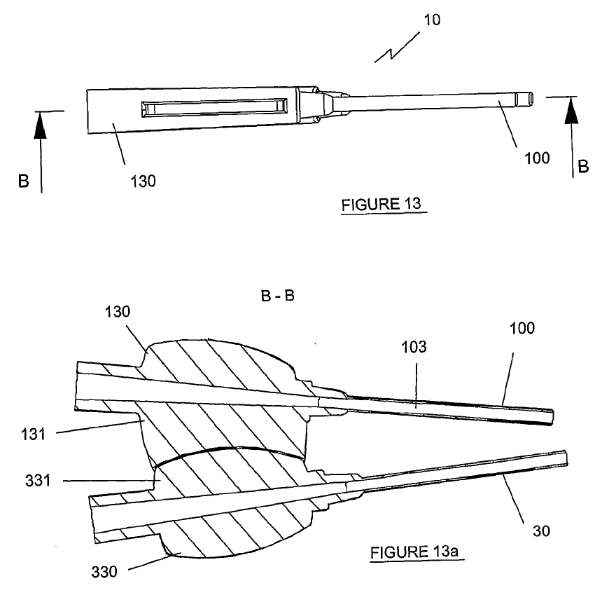

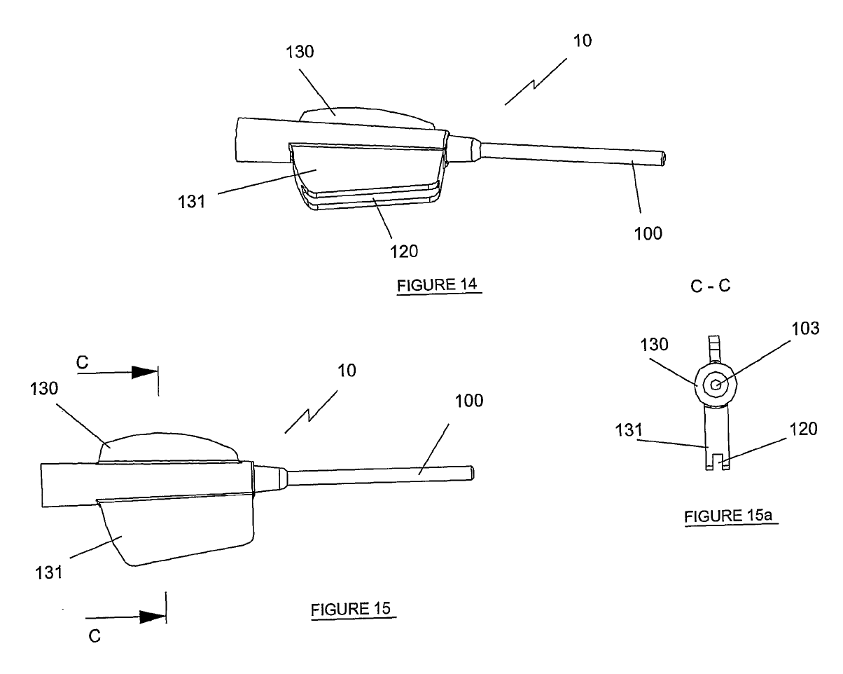

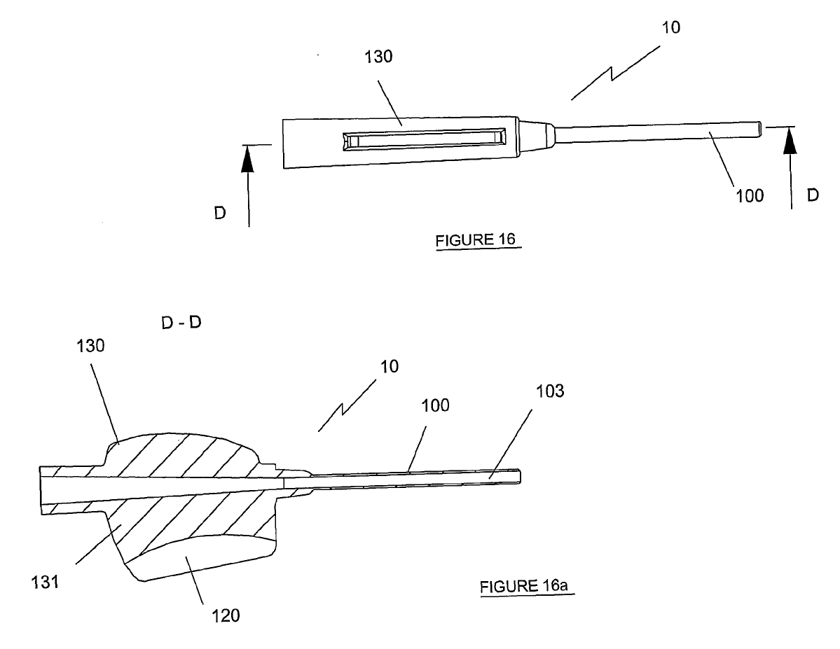

Referring now to FIG. 9, the drug delivery device of the invention, indicated generally by reference numeral 10 is shown. Drug delivery device 10 includes an elongate, flexible tube 100 having a distal end 101, a distal tip 104 and a proximal end 102. The drug delivery device 10 also includes a handle 130 positioned on the tube 100 at the proximal end 102 thereof. The tube 100 can be formed from an extruded plastic material such as polyamide. The proximal end 102 can be provided with a fitting (not shown) adapted to receive a pressurized liquid, which can comprise a medicament in liquid form, from a suitable source. The fitting communicates with lumen 103 of the tube 100 and the lumen 103 extends the entire length of the tube 100. The tube 100 is open at its distal tip 104 so that the tube 100 is capable of fluid communication with the guide wire lumen 46 of the catheter 30.

As shown in FIG. 10, the handle 130 of the drug delivery device 10 includes a recess 120 therein for removably receiving a wing 331 on the handle 330 of the MULTI-EXCHANGE™ catheter. The tube 100 of the drug delivery device 10 is attached to the handle 130 by gluing or alternatively the tube 100 may be moulded into the handle 130 during injection moulding.

Thus, the handle 130 on the proximal end 102 of the drug delivery device 10 is adapted to be fixedly coupled with the handle 330 in order to secure the drug delivery device 10 thereto. The handle 330 of the MULTI-EXCHANGE™ catheter has a wing 331 and the handle 130 of the drug delivery device 10 has a correspondingly sized and shaped recess 120 for receiving the wing 331. Both the recess 120 and wing 331 can be made from a resilient material to enable the handle 130 and the handle 330 to snap-fit together.

The flexible tube 100 is substantially circular in cross-section, but also may be non-circular; in either case, the cross-sectional dimensions of the tube 100 are smaller than the cross-sectional dimensions of the guide wire lumen 46 of the MULTI-EXCHANGE™ catheter such that the tube 100 is slidably movable longitudinally within the guide wire lumen 46 of the catheter 30.

The MULTI-EXCHANGE™ catheter 30 is provided with guide means 32 operable to permit the tube 100 to enter the catheter 30 via the guide member 32 and through the longitudinal guide way 48 of the catheter 30 and be advanced distally within the guide wire lumen 46 of the catheter 30.

With the guide member 32 mounted on the catheter 30, flaps 50 of the catheter 30 are spread apart on a length between the proximal and the distal ends of the guide member 32, but the flaps 50 remain close together outside the guide member 32. The distal end 101 of the tube 100 can then be inserted transversely into the guide wire lumen 46 of the catheter 30 via an opening in the guide member 32 and the spread-apart flaps 50. The tube 100 can then be advanced within the guide wire lumen 46 of the catheter 30 towards the distal end 38 of the catheter 30. The advancement of the tube 100 continues until the handle 130 of the drug delivery device 10 is aligned with the handle 330 of the catheter 30. With the handle 130 of the drug delivery device 10 and the handle 330 of the catheter 30 aligned, the wing 331 of the handle 330 is received within the correspondingly sized recess 120 such that the two handles 130, 330 are connected securely together. The length of the tube 100 from its point of exit 107 from the handle 130 to the distal tip 104 is such that when the handle 130 is aligned with the handle 330 of the catheter 30, the distal tip 104 is positioned under the balloon 40 close to the distal end 38 of the catheter 30 but not protruding out of the guide wire lumen 46. Thus, medicament in liquid form may be directed into the patient from a reservoir (not shown) through tube 100 and into the catheter 30 from when the medicament can be directed to the required site. A certain amount of backflow of the liquid exiting the tube 100 may occur during the process of drug infusion. The above described positioning of the distal tip 104 of the tube 100 avoids accidental leaking of the liquid medication out of the catheter 30 through the cut guide way 48 in the region indicated by A in FIG. 9 where the cut guide way 48 is present in the body of the catheter 30.

An example of a method of use of the drug delivery device of the invention will now be hereunder described.

After the PTCA procedure discussed above has been performed using the applicant's MULTI-EXCHANGE™ catheter 30, the balloon 40 is positioned across the lesion.

The guide wire 15 is removed from the guide wire lumen 46 of the catheter 30.

The guide member 32 is then positioned at any point along the proximal end 37 of the catheter 30, which is protruding out of the patient.

After that, a medication liquid is infused into the lumen 103 of the tube 100 by carrying out the following steps:
(a) The distal end 104 of the tube 100 is loaded via the guide member 32 into the guide wire lumen 46 and advanced through the guide wire lumen 46, towards the distal end 38 of the catheter 30. This can be done at whatever position the guide member 32 is along the catheter 30. The tube 100 is advanced until the handle 130 of the drug delivery device and handle 330 of the catheter are aligned side by side each other;
(b) Connecting the drug delivery device to the MULTI-EXCHANGE™ catheter 30 by aligning the handle 130 of the drug delivery device and the handle 330 of the catheter 30 by snap-fitting together the wing 131 of the handle and the recess 120 of the handle 130. At this stage, the tip 104 of the tube 100, as discussed above, is positioned under the balloon 40 of the catheter 30 close to the tip 38 of the catheter 30;
(c) The distal tip 38 of the catheter 30 is then drawn slightly proximally in order to position the tip 38 of the catheter at the site of the stenosis;
(d) At this stage the drug delivery device 10 is checked to ensure that there is no air in the tubing;
(e) A stopcock is attached to a first syringe and then onto the handle of the drug delivery device;
(f) a vacuum is pulled until blood is evident in the first syringe;
(g) the stopcock is closed and the first syringe is disconnected from the drug delivery device;
(h) a second syringe containing the medication is connected to the drug delivery device via the stopcock;
(i) the stopcock is opened and the medication from the second syringe is injected into the tube 100 of the drug delivery device 10;
(j) the stopcock is closed and the second syringe is disconnected; and
(k) optionally, the second syringe is filled with saline solution and the saline solution is injected through the drug delivery device to ensure all the medication has reached the desired site.

The medication liquid is then transported under a predetermined pressure to the distal tip 104 of the tube. The medication liquid exits the open ended tube 100 at its distal tip 104 and enters the guide wire lumen 46. This portion of the guide wire lumen 46 is defined by a continuous surrounding wall 49, as in FIG. 7, and, therefore, no leaking may occur during the advancement of the medication liquid in this portion of the guide wire lumen 46. The medication liquid exits the guide wire lumen 46 at its distal tip 38 and gently flushes the walls of the dilated blood vessel.

Upon completion of the drug delivery procedure, the catheter 30 together with the drug delivery device 10 can be removed from the blood vessel.

It is to be understood that the invention is not limited to the specific details described herein which are given by way of example only and that various modification and alterations are possible without departing from the scope of the invention as defined in the appended claims.

1. A drug delivery device comprising an elongate flexible tube having a proximal end and a distal end, the elongate flexible tube being adapted to be slidably movable in a lumen of a catheter and guide wire exchange system; the elongate flexible tube having a lumen extending therethrough, the lumen of the tube being open at the proximal end and distal end and the lumen being adapted to receive liquid under pressure; the drug delivery device also comprising means for engaging the drug delivery device with the catheter and guide wire exchange system, whereby, in use, the drug delivery device is in fluid communication with the catheter and guide wire exchange system and a medication in liquid form is delivered from a reservoir through the elongate flexible tube to the distal end thereof for delivery of the medication to a treatment site.

2. A drug delivery device as claimed in claim 1 wherein the elongate flexible tube is adapted to be slidably moveable in a guide wire lumen of the catheter and guide exchange system.

3. A drug delivery device as claimed in claim 1 or 2 wherein the catheter and guide wire exchange system includes a longitudinal guide way formed in the catheter shaft to enable transverse access to the lumen of the catheter, the guide way extending along a major portion of the length of the catheter shaft from a location adjacent the shaft proximal end to a distal terminal end proximal of the catheter shaft distal end, thereby defining an uncut distal segment of the catheter shaft.

4. A drug delivery device as claimed in any one of claims 2 or 3 wherein, in use, the elongate flexible tube of the drug delivery device is introduced through a guide member of the catheter and guide wire exchange system.

5. A drug delivery device as claimed in any preceding claim wherein the means for engaging the drug delivery device with the catheter and guide wire exchange system comprises a handle mounted on the proximal end of the elongate flexible tube and adapted for engagement with a handle of the catheter and guide wire exchange system.

6. A drug delivery device as claimed in claim 5 wherein the handle of the drug delivery device includes a recess which is correspondingly sized and shaped to engageably receive a portion of the handle of the catheter.

7. A drug delivery device as claimed in claim 5 or 6 wherein the handle of the drug delivery device and the handle of the catheter are resiliently formed so that the handles are connected together by snap-fitting the catheter handle into engagement with the drug delivery device handle.

8. A method of preparing a liquid medicament for delivery, comprising the following steps:
(a) loading, via a guide member, a distal end of a drug delivery device comprising an elongate flexible tube into a lumen of a catheter and guide wire exchange system and advancing the distal end of the elongate flexible tube towards the distal end of the catheter;
(b) advancing the tube until a handle of the drug delivery device and handle of the catheter are aligned side by side each other,
(c) connecting the drug delivery device to the catheter by connecting the handle of the drug delivery device with the handle of the catheter, and
(d) providing a reservoir for liquid medicament and directing medicament from the reservoir to the distal ends of the drug delivery device and catheter.

9. A method as claimed in claim 8 wherein at step (a) the elongate flexible tube is loaded into a guide wire lumen of the catheter.

10. A method as claimed in claims 8 or 9 wherein between step (c) and step (d), there is included a step of drawing the distal tip of the catheter proximally in order to position the tip of the catheter at a desired site.

11. A method of delivering a drug, comprising the following steps:
(A) loading, via a guide member, a distal end of a drug delivery device comprising an elongate flexible tube into a lumen of a catheter and guide wire exchange system and advancing the distal end of the elongate flexible tube towards the distal end of the catheter,
(B) advancing the tube until a handle of the drug delivery device and handle of the catheter are aligned side by side each other,
(C) connecting the drug delivery device to the catheter by connecting the handle of the drug delivery device with the handle of the catheter, and
(D) injecting a medication in liquid form from a reservoir through the drug delivery device so that the medicament is delivered through the drug delivery device to the required treatment site.

12. A method as claimed in claim 11 wherein at step (A), the elongate flexible tube is loaded into a guide wire lumen of the catheter.

13. A method as claimed in claims 11 wherein between step (C) and (D) there is included a step of drawing the distal tip of the catheter proximally in order to position the tip of the catheter at a treatment site.

14. A method as claimed in claim 11 wherein the method includes the following step: after injecting the medication using the drug delivery device at step (D), flushing the device with saline solution and injecting the saline solution through the drug delivery device to ensure all the medication has reached the treatment site.
